(19) United States
(12) Patent Application Publication
(10) Pub. No.: US 2015/0196216 A1
(43) Pub. Date: Jul. 16, 2015

(54) MEDICAL DEVICES FOR MAPPING CARDIAC TISSUE

(71) Applicant: BOSTON SCIENTIFIC SCIMED, INC., Maple Grove, MN (US)

(72) Inventors: JACOB I. LAUGHNER, ST. PAUL, MN (US); SHIBAJI SHOME, ARDEN HILLS, MN (US); KEVIN J. STALSBERG, WHITE BEAR LAKE, MN (US); SCOTT A. MEYER, LAKEVILLE, MN (US)

(73) Assignee: BOSTON SCIENTIFIC SCIMED, INC., MAPLE GROVE, MN (US)

(21) Appl. No.: 14/595,656

(22) Filed: Jan. 13, 2015

Related U.S. Application Data
(60) Provisional application No. 61/926,750, filed on Jan. 13, 2014.

(51) Int. Cl. A61B 5/042; A61B 5/044; A61B 5/00; A61B 5/04

(52) U.S. Cl. CPC A61B 5/0422; A61B 5/04012; A61B 5/044; A61B 5/6858

(57) ABSTRACT

Medical devices and methods for making and using medical devices are disclosed. An example medical device may include a catheter shaft with a plurality of electrodes coupled thereto and a processor coupled to the catheter shaft. The processor may be capable of collecting a set of signals from the plurality of electrodes, generating a set of data from at least one of the set of signals, wherein the data set includes at least one known data point and one or more unknown data points, determining a non-linear distance between the at least one known data point and the one or more unknown data points, and assigning a value to at least one of the unknown data points.

### CROSS-REFERENCE TO RELATED APPLICATIONS

This application claims priority under 35 U.S.C. §119 to U.S. Provisional Application Ser. No. 61/926,750 filed Jan. 13, 2014, the entirety of which is incorporated herein by reference.

### TECHNICAL FIELD

The present disclosure pertains to medical devices, and methods for manufacturing medical devices. More particularly, the present disclosure pertains to medical devices and methods for mapping and/or ablating cardiac tissue.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

The invention provides design, material, manufacturing method, and use alternatives for medical devices. An example medical device is disclosed. The medical device comprises:

a catheter shaft with a plurality of electrodes coupled thereto;

a processor coupled to the catheter shaft, wherein the processor is capable of:
collecting a set of signals from the plurality of electrodes;
generating a data set from at least one of the set of signals, wherein the data set includes at least one known data point and one or more unknown data points;
determining a non-linear distance between the at least one known data point and the one or more unknown data points; and
assigning a value to at least one of the unknown data points.

Additionally or alternatively to any of the examples above, wherein collecting the set of signals further includes sensing a change in electrical potential by any one of the plurality of electrodes.

Additionally or alternatively to any of the examples above, further comprising identifying a threshold value corresponding to a minimum change in electrical potential by any one of the plurality of electrodes and wherein collecting the set of signals includes collecting only those signals that are above the threshold value.

Additionally or alternatively to any of the examples above, wherein collecting the set of signals includes determining an activation time at one or more of the plurality of electrodes.

Additionally or alternatively to any of the examples above, wherein determining the activation time includes identifying a fiducial point corresponding to a change in electrical potential and determining a time latency between a reference point and the fiducial point.

Additionally or alternatively to any of the examples above, wherein determining the non-linear distance between at least one known data point and one or more unknown data points includes creating a mesh of interconnected nodes between the known data points, the unknown data points or both the known and unknown data points.

Additionally or alternatively to any of the examples above, wherein determining the non-linear distance between at least one known data point and one or more unknown data points includes creating a triangular mesh between the known data points, the unknown data points or both the known and unknown data points.

Additionally or alternatively to any of the examples above, wherein determining the non-linear distance between at least one known data point and one or more unknown data points includes upsampling the mesh of interconnected nodes.

Additionally or alternatively to any of the examples above, wherein determining the non-linear distance between at least one known data point and one or more unknown data points includes determining a geodesic distance between the known data points, unknown data points or both the known and unknown data points.

Additionally or alternatively to any of the examples above, wherein assigning a value to at least one of the unknown data points includes interpolating the at least one unknown data point, and wherein interpolating the at least one unknown data point includes utilizing the non-linear distance between the at least one known data point and the unknown data point.

Additionally or alternatively to any of the examples above, wherein interpolating the at least one unknown data point includes radial basis function interpolation.

Additionally or alternatively to any of the examples above, wherein interpolating the at least one unknown data point further includes utilizing a geodesic distance in the radial basis function interpolation.

Additionally or alternatively to any of the examples above, wherein utilizing the non-linear distance between the at least one known data point and the unknown data point further comprises using a geodesic distance between the at least one known data point and the unknown data point.

Additionally or alternatively to any of the examples above, wherein assigning a value to at least one of the unknown data points includes assigning an activation time to at least one of the unknown data points.

Additionally or alternatively to any of the examples above, wherein assigning an activation time to at least one of the unknown data points further comprises radial basis function interpolation of activation times, wherein radial basis function interpolation incorporates a geodesic distance between at least one known data point and one or more unknown data points.

Additionally or alternatively to any of the examples above, further comprising generating a visual display of between at least one known data point, one or more unknown data points or both.

Additionally or alternatively to any of the examples above, wherein generating a visual representation includes creating an activation map.

Additionally or alternatively to any of the examples above, wherein the activation map further comprises a plurality of color indicators.

A method for delivering a medical device is disclosed. The method comprises:

delivering the medical device of any one of claims 1-18 into the heart of a patient.

A medical device for mapping the electrical activity of the heart is disclosed. The medical device comprises:
a catheter shaft coupled to a sensing element, wherein the sensing element includes a plurality of electrodes coupled thereto;
a processor coupled to the catheter shaft, wherein the processor is capable of:
   collecting a set of signals from the plurality of electrodes;
   generating a data set from at least one of the set of signals, wherein the data set includes at least one known data point and one or more unknown data points;
   determining a non-linear distance between the at least one known data point and the one or more unknown data points;
   interpolating the at least one unknown data point from the at least one known data point, wherein interpolating the at least one unknown data point includes utilizing the non-linear distance between the at least one known data point and the unknown data point; and
   assigning a value to at least one of the unknown data points.

Additionally or alternatively to any of the examples above, wherein collecting the set of signals further includes sensing a change in electrical potential by any one of the plurality of electrodes.

Additionally or alternatively to any of the examples above, further comprising identifying a threshold value corresponding to a minimum change in electrical potential by any one of the plurality of electrodes and wherein collecting the set of signals includes collecting only those signals that are above the threshold value.

Additionally or alternatively to any of the examples above, wherein collecting the set of signals includes determining an activation time at one or more of the plurality of electrodes.

Additionally or alternatively to any of the examples above, wherein determining the activation time includes identifying a fiducial point corresponding to a change in electrical potential and determining a time latency between a reference point and the fiducial point.

Additionally or alternatively to any of the examples above, wherein determining the non-linear distance between at least one known data point and one or more unknown data points includes creating a mesh of interconnected nodes between the known data points, the unknown data points or both the known and unknown data points.

Additionally or alternatively to any of the examples above, wherein determining the non-linear distance between at least one known data point and one or more unknown data points includes creating a triangular mesh between the known data points, the unknown data points or both the known and unknown data points.

Additionally or alternatively to any of the examples above, wherein determining the non-linear distance between at least one known data point and one or more unknown data points includes upsampling the mesh of interconnected nodes.

Additionally or alternatively to any of the examples above, wherein determining the non-linear distance between at least one known data point and one or more unknown data points includes determining a geodesic distance between the known data points, unknown data points or both the known and unknown data points.

Additionally or alternatively to any of the examples above, wherein interpolating the at least one unknown data point includes Kriging interpolation.

Additionally or alternatively to any of the examples above, wherein interpolating the at least one unknown data point includes radial basis function interpolation.

Additionally or alternatively to any of the examples above, wherein interpolating the at least one unknown data point further includes utilizing a geodesic distance in the radial basis function interpolation.

Additionally or alternatively to any of the examples above, wherein utilizing the non-linear distance between the at least one known data point and the unknown data point further comprises using a geodesic distance between the at least one known data point and the unknown data point.

Additionally or alternatively to any of the examples above, wherein assigning a value to at least one of the unknown data points includes assigning an activation time to at least one of the unknown data points.

Additionally or alternatively to any of the examples above, wherein assigning an activation time to at least one of the unknown data points further comprises radial basis function interpolation of activation times, wherein radial basis function interpolation incorporates a geodesic distance between at least one known data point and one or more unknown data points.

Additionally or alternatively to any of the examples above, further comprising generating a visual display of between at least one known data point, one or more unknown data points or both.

Additionally or alternatively to any of the examples above, wherein generating a visual representation includes creating an activation map.

A method of mapping the electrical activity of the heart is disclosed. The method comprises:

advancing a catheter shaft with a plurality of electrodes coupled thereto into a chamber of the heart, wherein the catheter shaft is coupled to a processor, wherein the processor is capable of:
collecting a set of signals from the plurality of electrodes;
generating a data set from at least one of the set of signals, wherein the data set includes at least one known data point and one or more unknown data points;
determining a non-linear distance between the at least one known data point and the one or more unknown data points; and
assigning a value to at least one of the unknown data points.

Additionally or alternatively to any of the examples above, wherein collecting the set of signals further includes sensing a change in electrical potential by any one of the plurality of electrodes.

Additionally or alternatively to any of the examples above, further comprising identifying a threshold value corresponding to a minimum change in electrical potential by any one of the plurality of electrodes and wherein collecting the set of signals includes collecting only those signals that are above the threshold value.

Additionally or alternatively to any of the examples above, wherein collecting the set of signals includes determining an activation time at one or more of the plurality of electrodes.

Additionally or alternatively to any of the examples above, wherein determining the activation time includes identifying a fiducial point corresponding to a change in electrical potential and determining a time latency between a reference point and the fiducial point.

Additionally or alternatively to any of the examples above, wherein determining the non-linear distance between at least one known data point and one or more unknown data points includes creating a mesh of interconnected nodes between the known data points, the unknown data points or both the known and unknown data points.

Additionally or alternatively to any of the examples above, wherein determining the non-linear distance between at least one known data point and one or more unknown data points includes creating a triangular mesh between the known data points, the unknown data points or both the known and unknown data points.

Additionally or alternatively to any of the examples above, wherein determining the non-linear distance between at least one known data point and one or more unknown data points includes upsampling the mesh of interconnected nodes.

Additionally or alternatively to any of the examples above, wherein determining the non-linear distance between at least one known data point and one or more unknown data points includes determining a geodesic distance between the known data points, unknown data points or both the known and unknown data points.

Additionally or alternatively to any of the examples above, wherein assigning a value to at least one of the unknown data points includes interpolating the at least one unknown data point, and wherein interpolating the at least one unknown data point includes utilizing the non-linear distance between the at least one known data point and the unknown data point.

Additionally or alternatively to any of the examples above, wherein interpolating the at least one unknown data point includes radial basis function interpolation.

Additionally or alternatively to any of the examples above, wherein interpolating the at least one unknown data point further includes utilizing a geodesic distance in the radial basis function interpolation.

Additionally or alternatively to any of the examples above, wherein utilizing the non-linear distance between the at least one known data point and the unknown data point further comprises using a geodesic distance between the at least one known data point and the unknown data point.

Additionally or alternatively to any of the examples above, wherein assigning a value to at least one of the unknown data points includes assigning an activation time to at least one of the unknown data points.

Additionally or alternatively to any of the examples above, wherein assigning an activation time to at least one of the unknown data points further comprises radial basis function interpolation of activation times, wherein radial basis function interpolation incorporates a geodesic distance between at least one known data point and one or more unknown data points.

Additionally or alternatively to any of the examples above, further comprising generating a visual display of between at least one known data point, one or more unknown data points or both.

Additionally or alternatively to any of the examples above, wherein generating a visual representation includes creating an activation map.

Additionally or alternatively to any of the examples above, wherein the activation map further comprises a plurality of color indicators.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an example catheter system for accessing a targeted tissue region in the body for diagnostic and therapeutic purposes.

FIG. 2 is a schematic view of an example mapping catheter having a basket functional element carrying structure for use in association with the system of FIG. 1.

FIG. 3 is a schematic view of an example functional element including a plurality of mapping electrodes.

FIG. 4 is an illustration of an example activation map displaying known and unknown activation times.

FIG. 5 is an illustration of an example electrode mesh.

FIG. 6 is an illustration of an example upsampled electrode mesh.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Mapping the electrophysiology of heart rhythm disorders often involves the introduction of a constellation catheter or other mapping/sensing device having a plurality of sensors into a cardiac chamber. The sensors detect the electric activity of the heart at sensor locations. It may be desirable to have the electric activity processed into electrogram signals that accurately represent cellular excitation through cardiac tissue relative to the sensor locations. A processing system may then analyze and output the signal to a display device. Further, the processing system may output the signal as an activation or vector field map. The physician may use the activation or vector field map to perform a diagnostic procedure.

However, in some cases the sensing electrodes may fail to accurately detect the electrical activity of heart. The failure of the electrodes to detect a signal may limit the ability of the processing system to accurately display information used for diagnostic procedures. For example, an activation map may be generated that contains missing information and/or inaccurate visual representations. Therefore, it may be desirable to replace poor or non-existent electrical signal information with information that is believed to be accurate. In some instances, interpolation may be used to replace poor/missing data. Standard interpolation methods may have limitations due to both the temporal nature of the activation signals and the three-dimensional spatial configuration of sensing electrodes located in an anatomical region. The methods and systems disclosed herein are designed to overcome at least some of the limitations of standard interpolation methods used to interpolate poor or non-existent activation signals. For example, some of the methods disclosed herein may utilize geodesic distance calculations in order to improve the accuracy of interpolation methods. Other methods and medical devices are also disclosed.

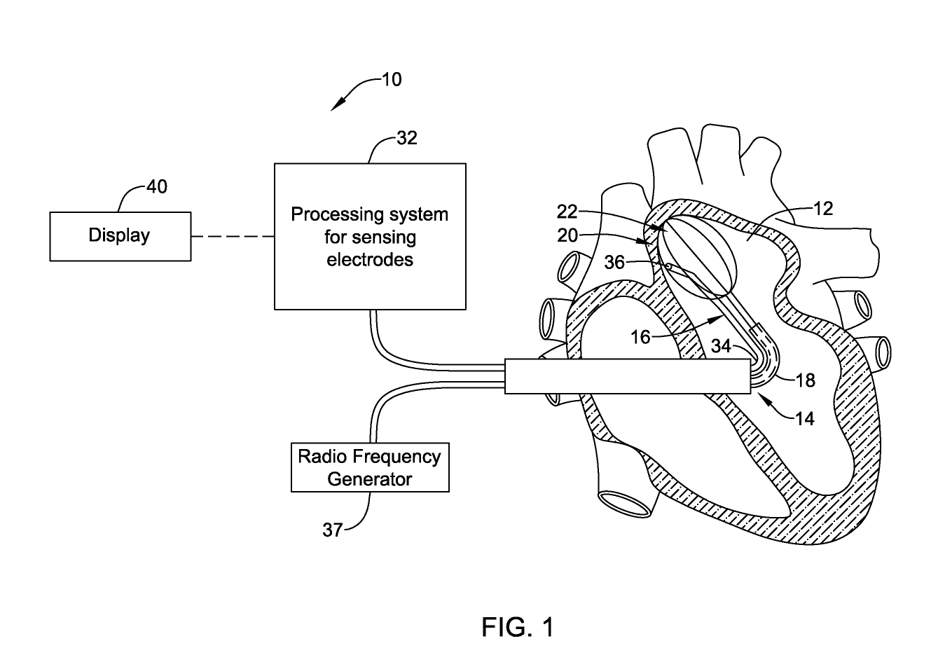

FIG. 1 is a schematic view of a system 10 for accessing a targeted tissue region in the body for diagnostic and/or therapeutic purposes. FIG. 1 generally shows the system 10 deployed in the left atrium of the heart. Alternatively, system 10 can be deployed in other regions of the heart, such as the left ventricle, right atrium, or right ventricle. While the illustrated embodiment shows the system 10 being used for ablating myocardial tissue, the system 10 (and the methods described herein) may alternatively be configured for use in other tissue ablation applications, such as procedures for ablating tissue in the prostrate, brain, gall bladder, uterus, nerves, blood vessels and other regions of the body, including in systems that are not necessarily catheter-based.

The system 10 includes a mapping probe 14 and an ablation probe 16. Each probe 14/16 may be separately introduced into the selected heart region 12 through a vein or artery (e.g., the femoral vein or artery) using a suitable percutaneous access technique. Alternatively, the mapping probe 14 and ablation probe 16 can be assembled in an integrated structure for simultaneous introduction and deployment in the heart region 12.

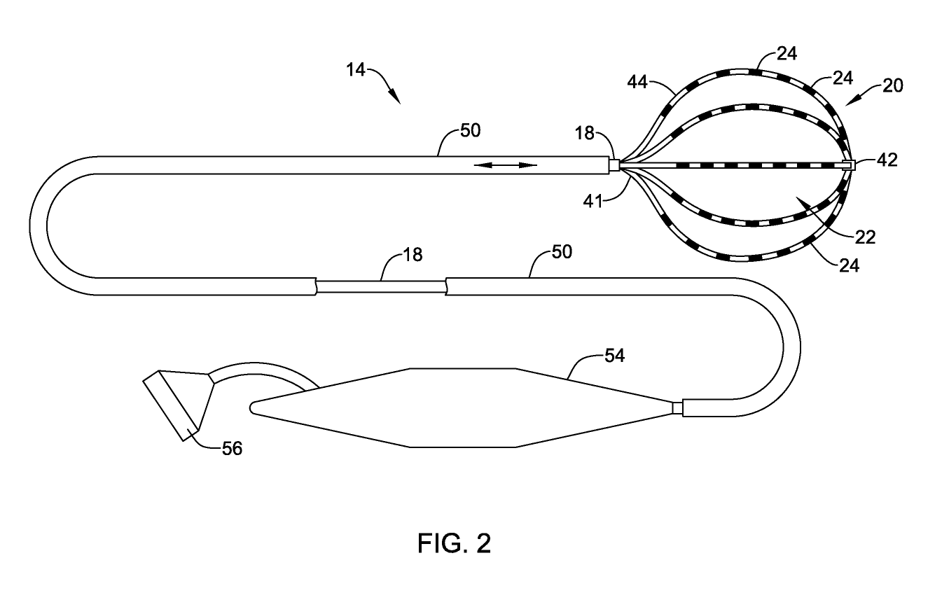

The mapping probe 14 may have a flexible catheter body 18. The distal end of the catheter body 18 carries a three-dimensional multiple electrode structure 20. In the illustrated embodiment, the structure 20 takes the form of a basket defining an open interior space 22 (see FIG. 2), although other multiple electrode structures could be used. The multiple electrode structure 20 carries a plurality of mapping electrodes 24 (not explicitly shown on FIG. 1, but shown on FIG. 2) each having an electrode location on structure 20 and a conductive member. Each electrode 24 may be configured to sense intrinsic physiological activity in the anatomical region. In some embodiments, the electrodes 24 may be configured to detect activation signals of the intrinsic physiological activity within the anatomical structure (e.g., the activation times of cardiac activity).

The electrodes 24 are electrically coupled to a processing system 32. A signal wire (not shown) may be electrically coupled to each electrode 24 on the basket structure 20. The wires may extend through the body 18 of the probe 14 and electrically couple each electrode 24 to an input of the processing system 32. The electrodes 24 sense electrical activity in the anatomical region, e.g., myocardial tissue. The sensed activity (e.g., activation signals) may be processed by the processing system 32 to assist the physician by generating an anatomical map (e.g., a vector field map, an activation time map) to identify the site or sites within the heart appropriate for a diagnostic and/or treatment procedure, e.g. an ablation procedure. For example, the processing system 32 may identify a near-field signal component (e.g., activation signals originating from cellular tissue adjacent to the mapping electrode 24) or from an obstructive far-field signal component (e.g., activation signals originating from non-adjacent tissue). For example, the near-field signal component may include activation signals originating from atrial myocardial tissue whereas the far-field signal component may include activation signals originating from ventricular myocardial tissue. The near-field activation signal component may be further analyzed to find the presence of a pathology and to determine a location suitable for ablation for treatment of the pathology (e.g., ablation therapy).

The processing system 32 may include dedicated circuitry (e.g., discrete logic elements and one or more microcontrollers; application-specific integrated circuits (ASICs); or specially configured programmable devices, such as, for example, programmable logic devices (PLDs) or field programmable gate arrays (FPGAs)) for receiving and/or processing the acquired activation signals. In some embodiments, the processing system 32 includes a general purpose microprocessor and/or a specialized microprocessor (e.g., a digital signal processor, or DSP, which may be optimized for processing activation signals) that executes instructions to receive, analyze and display information associated with the received activation signals. In such implementations, the processing system 32 can include program instructions, which when executed, perform part of the signal processing. Program instructions can include, for example, firmware, microcode or application code that is executed by microprocessors or microcontrollers. The above-mentioned implementations are merely exemplary, and the reader will appreciate that the processing system 32 can take any suitable form.

In some embodiments, the processing system 32 may be configured to measure the electrical activity in the myocardial tissue adjacent to the electrodes 24. For example, in some embodiments, the processing system 32 is configured to detect electrical activity associated with a dominant rotor or divergent activation pattern in the anatomical feature being mapped. For example, dominant rotors and/or divergent activation patterns may have a role in the initiation and maintenance of atrial fibrillation, and ablation of the rotor path, rotor core, and/or divergent foci may be effective in terminating the atrial fibrillation. In either situation, the processing system 32 processes the sensed activation signals to generate a display of relevant characteristics, such as an isochronal map, activation time map, action potential duration (APD) map, a vector field map, a contour map, a reliability map, an electrogram, a cardiac action potential and the like. The relevant characteristics may be used by the physician to identify a site suitable for ablation therapy.

The ablation probe 16 includes a flexible catheter body 34 that carries one or more ablation electrodes 36. The one or more ablation electrodes 36 are electrically connected to a radio frequency (RF) generator 37 that is configured to deliver ablation energy to the one or more ablation electrodes 36. The ablation probe 16 may be movable with respect to the anatomical feature to be treated, as well as the structure 20. The ablation probe 16 may be positionable between or adjacent to electrodes 24 of the structure 20 as the one or more ablation electrodes 36 are positioned with respect to the tissue to be treated.

The processing system 32 may output data to a suitable output or display device 40, which may display relevant information for a clinician. In the illustrated embodiment, device 40 is a CRT, LED, or other type of display, or a printer. Device 40 presents the relevant characteristics in a format most useful to the physician. In addition, processing system 32 may generate position-identifying output for display on device 40 that aids the physician in guiding ablation electrode(s) 36 into contact with tissue at the site identified for ablation.

FIG. 2 illustrates mapping catheter 14 and shows electrodes 24 at the distal end suitable for use in the system 10 shown in FIG. 1. Mapping catheter 14 may have a flexible catheter body 18, the distal end of which may carry three dimensional structure 20 with mapping electrodes or sensors 24. Mapping electrodes 24 may sense electrical activity (e.g., activation signals) in the myocardial tissue. The sensed activity may be processed by the processing system 32 to assist the physician in identifying the site or sites having a heart rhythm disorder or other myocardial pathology via generated and displayed relevant characteristics. This information can then be used to determine an appropriate location for applying appropriate therapy, such as ablation, to the identified sites, and to navigate the one or more ablation electrodes 36 to the identified sites.

The illustrated three-dimensional structure 20 comprises a base member 41 and an end cap 42 between which flexible splines 44 generally extend in a circumferentially spaced relationship. As discussed herein, the three dimensional structure 20 may take the form of a basket defining an open interior space 22. In some embodiments, the splines 44 are made of a resilient inert material, such as Nitinol, other metals, silicone rubber, suitable polymers, or the like and are connected between the base member 41 and the end cap 42 in a resilient, pretensioned condition, to bend and conform to the tissue surface they contact. In the illustrated embodiment, eight splines 44 form the three dimensional structure 20. Additional or fewer splines 44 could be used in other embodiments. As illustrated, each spline 44 carries eight mapping electrodes 24. Additional or fewer mapping electrodes 24 could be disposed on each spline 44 in other embodiments of the three dimensional structure 20. In the illustrated embodiment, the three dimensional structure 20 is relatively small (e.g., 40 mm or less in diameter). In alternative embodiments, the three dimensional structure 20 is even smaller or larger (e.g., 40 mm in diameter or greater).

A slidable sheath 50 may be movable along the major axis of the catheter body 18. Moving the sheath 50 distally relative to catheter body 18 may cause sheath 50 to move over the three dimensional structure 20, thereby collapsing the structure 20 into a compact, low profile condition suitable for introduction into and/or removal from an interior space of an anatomical structure, such as, for example, the heart. In contrast, moving the sheath 50 proximally relative to the catheter body may expose the three dimensional structure 20, allowing the structure 20 to elastically expand and assume the pretensed position illustrated in FIG. 2.

A signal wire (not shown) may be electrically coupled to each mapping electrode 24. The wires may extend through the body 18 of the mapping catheter 20 (or otherwise through and/or along the body 18) into a handle 54, in which they are coupled to an external connector 56, which may be a multiple pin connector. The connector 56 electrically couples the mapping electrodes 24 to the processing system 32. These are just examples. Some addition details regarding these and other example mapping systems and methods for processing signals generated by the mapping catheter can be found in U.S. Pat. Nos. 6,070,094, 6,233,491, and 6,735,465, the disclosures of which are hereby expressly incorporated herein by reference.

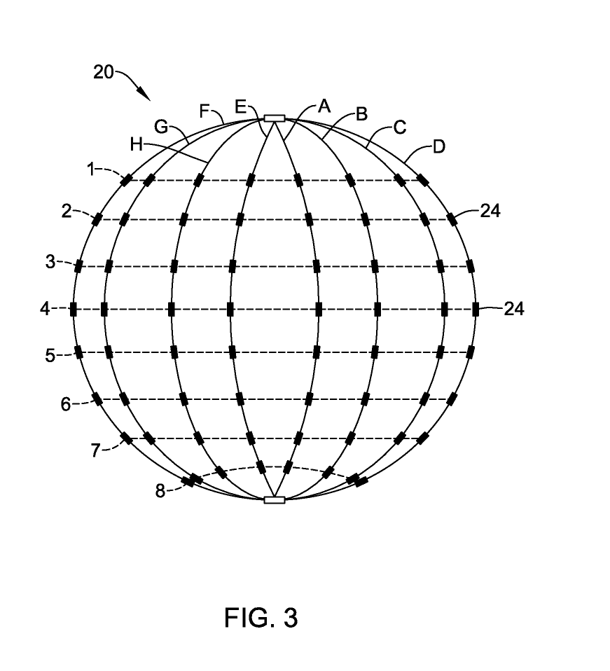

To illustrate the operation of the system 10, FIG. 3 is a schematic side view of an embodiment of the basket structure 20 including a plurality of mapping electrodes 24. In the illustrated embodiment, the basket structure includes 64 mapping electrodes 24. The mapping electrodes 24 are disposed in groups of eight electrodes (labeled 1, 2, 3, 4, 5, 6, 7, and 8) on each of eight splines (labeled A, B, C, D, E, F, G, and H). While an arrangement of sixty-four mapping electrodes 24 is shown disposed on a basket structure 20, the mapping electrodes 24 may alternatively be arranged in different numbers (more or fewer splines and/or electrodes), on different structures, and/or in different positions. In addition, multiple basket structures can be deployed in the same or different anatomical structures to simultaneously obtain signals from different anatomical structures.

After the basket structure 20 is positioned adjacent to the anatomical structure to be treated (e.g. left atrium, left ventricle, right atrium, or right ventricle of the heart), the processing system 32 is configured to record the activation signals from each electrode 24 channel related to physiological activity of the anatomical structure (e.g., the electrodes 24 measure electrical activation signals associated with the physiology of the anatomical structure). The activation signals of physiological activity may be sensed in response to intrinsic physiological activity or based on a predetermined pacing protocol instituted by at least one of the plurality of electrodes 24.

The arrangement, size, spacing and location of electrodes along a constellation catheter or other mapping/sensing device, in combination with the specific geometry of the targeted anatomical structure, may contribute to the ability (or inability) of electrodes 24 to sense, measure, collect and transmit electrical activity of cellular tissue. As stated, because splines 44 of a mapping catheter, constellation catheter or other similar sensing device are bendable, they may conform to a specific anatomical region in a variety of shapes and/or configurations. Further, at any given position in the anatomical region, the electrode basket structure 20 may be manipulated such that one or more splines 44 may not contact adjacent cellular tissue. For example, splines 44 may twist, bend or lie atop one another, thereby separating splines 44 from nearby cellular tissue. Additionally, because electrodes 24 are disposed on one or more of splines 44, they also may not maintain contact with adjacent cellular tissue. Electrodes 24 that do not maintain contact with cellular tissue may be incapable of sensing, measuring, collecting and/or transmitting electrical activity information. Further, because electrodes 24 may be incapable of sensing, measuring, collecting and/or transmitting electrical activity information, processing system 32 may be incapable of accurately displaying diagnostic information. For example, some necessary information may be missing and/or displayed inaccurately.

In addition to that stated above, electrodes 24 may not be in contact with adjacent cellular tissue for other reasons. For example, manipulation of mapping catheter 14 may result in movement of electrodes 24, thereby creating poor electrode-to-tissue contact. Further, electrodes 24 may be positioned adjacent fibrous, dead or functionally refractory tissue. Electrodes 24 positioned adjacent fibrous, dead or functionally refractory tissue may not be able to sense changes in electrical potential because fibrous, dead or functionally refractory tissue may be incapable of depolarizing and/or responding to changes in electrical potential. Finally, far-field ventricular events and electrical line noise may distort measurement of tissue activity.

However, electrodes 24 that contact healthy, responsive cellular tissue may sense a change in the voltage potential of a propagating cellular activation wavefront. Further, in a normal functioning heart, electrical discharge of the myocardial cells may occur in a systematic, linear fashion. Therefore, detection of non-linear propagation of the cellular excitation wavefront may be indicative of cellular firing in an abnormal fashion. For example, cellular firing in a rotating pattern may indicate the presence of dominant rotors and/or divergent activation patterns. Further, because the presence of the abnormal cellular firing may occur over localized target tissue regions, it is possible that electrical activity may change form, strength or direction when propagating around, within, among or adjacent to diseased or abnormal cellular tissue. Identification of these localized areas of diseased or abnormal tissue may provide a clinician with a location for which to perform a therapeutic and/or diagnostic procedure. For example, identification of an area including reentrant or rotor currents may be indicative of an area of diseased or abnormal cellular tissue. The diseased or abnormal cellular tissue may be targeted for an ablative procedure. An activation time map 72 may be used to identify areas of circular, adherent, rotor or other abnormal cellular excitation wavefront propagation.

An activation map 72 may include a two-dimensional grid that visually represents mapping electrodes 24 located on a three dimensional mapping catheter (e.g. constellation catheter or other similar sensing device). For example, activation map 72 may include an 8×8 matrix displaying sixty-four (64) electrode spaces that represent the sixty-four (64) electrodes on a constellation catheter or similar sensing device. Mapping electrodes 24 may be organized and/or identified by electrode number (e.g. electrodes 1-8) and spline location (e.g. splines A-H). Other combinations of electrodes and/or splines are contemplated.

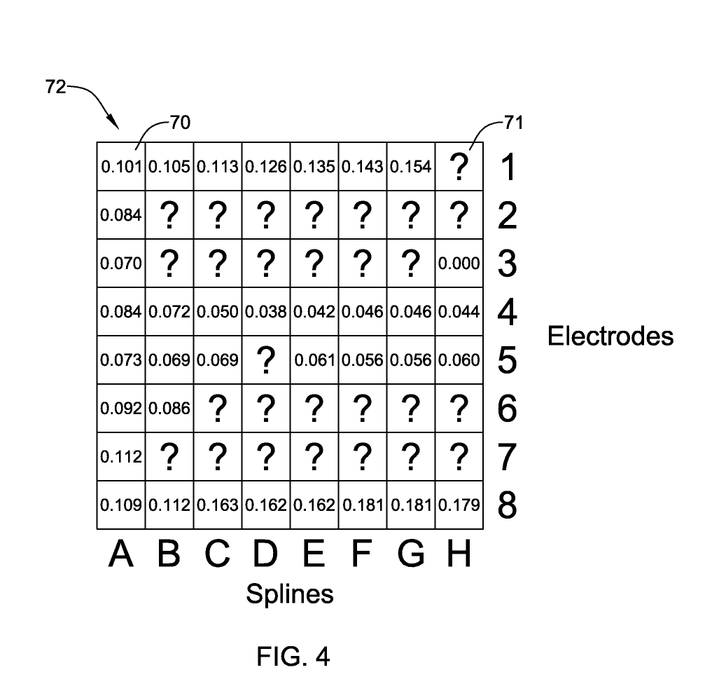

FIG. 4 illustrates an example activation map 72 showing activation times sensed by electrodes 24. In this example, activation map 72 takes the form of a grid that is designed to display activation times for all 64 electrodes 24 of multiple electrode structure 20. The activation time for an electrode 24 may be defined as the time elapsed between an activation “event” being sensed on a target mapping electrode 24 and a reference electrode. For example, a space 70 on map 72 representing electrode 1 on spline A displays an activation time of 0.101 ms. However, it is possible that one or more electrodes 24 will be unable to sense and/or collect an activation time. For example, one or more spaces like a space 71 representing electrode 1 on spline H may display a “?.” The “?” may indicate that the particular electrode corresponding to that location on the multiple electrode structure 20 cannot sense an activation time. Therefore, the “?” may represent missing signal data. Missing signal data and/or an incomplete activation map may prevent the identification of diseased or abnormal cellular tissue.

Another embodiment of the invention may include generating a color map corresponding to activation map 72. Each unique activation time may be assigned a unique, differentiating color. It is contemplated that a variety of color combinations may be included in generating the color-based activation time map. Further, the color map may be displayed on a display. Additionally, the color map may help a clinician identify the propagation direction of cellular firing. Activation map 72 may display an activation time or color for known signals and not display an activation time or color for unknown and/or missing activation time data. The use of color to differentiate activation times is just an example. It is contemplated that other means may be used to differentiate activation times. For example, texture, symbols, numbers, or the like may be used as differentiating characteristics.

In order to maximize the utility of activation map 72, it may be desirable to populate unknown activation times. Therefore, in some embodiments it may be desirable to interpolate activation times for missing signal data and populate and/or fill in the activation time map 72 accordingly. In practice, it may be that electrodes 24 in close proximity to one another will experience similar cellular events (e.g. depolarization). For example, as a cellular activation wavefront propagates across an atrial surface, electrodes 24 in close proximity to one another will likely experience similar cellular activation times. Therefore, when selecting an interpolation method, it may be desirable to select a method that incorporates the relative distance between neighboring electrodes and utilizes those distances in an algorithm to estimate unknown data points. One method to interpolate activation times and thereby fill in missing electrode data is to utilize an interpolation method that estimates the missing electrode data based on the electrode's relationship and/or proximity to known electrode data. The method may include identifying the physical position of all electrodes 24 in three-dimensional space, determining the distance between electrodes 24, and interpolating and/or estimating the missing electrode values. The estimated values may then be used to populate diagnostic displays (e.g. activation map). Therefore, the interpolation method may include any interpolation method that incorporates neighboring electrode information (e.g. distance between electrodes) in its estimation algorithm. Example interpolation methods may include Radial Basis Function (RBF) and/or Kriging interpolation. These are only examples. It is contemplated that other interpolation methods that incorporate neighboring data point information may be utilized with the embodiments disclosed herein.

As indicated above, some interpolation methods may incorporate the distance between electrodes as an input variable of their interpolation algorithm. For example, RBF and Kriging interpolation methods may incorporate the linear distance between unknown and known electrodes in their interpolation algorithms. The linear distance may be determined by calculating the “straight line” or “Euclidean” distance between electrodes 24. In non-curved space, it is generally understood that the shortest distance between two points is a straight line.

When collecting and analyzing the electrical activity of the heart, it is often desirable to collect and/or analyze the electrical activity as it is expressed and/or propagated through an anatomical region. It is generally understood that the anatomical shape of the interior walls of the heart are curved spaces. Further, because multiple electrode structure 20 may conform to the anatomical space in which it is deployed (e.g. heart chamber), electrodes 24 disposed on multiple electrode structure 20 may similarly conform to the anatomical space in which multiple electrode structure 20 is deployed. In practice, multiple electrode structure 20 is often deployed along the curved surface of an atrial chamber. In some embodiments it may be desirable to collect and/or analyze electrical activity as it occurs along the curved surface of an atrial chamber. Therefore, when incorporating the distance between electrodes into an interpolation method, it is often desirable to use the distance between the electrodes along the curved surface of the cardiac chamber. In contrast, it is often less desirable to calculate the linear distance between electrodes through open space and/or blood. Further, assuming a fixed distance between electrodes and/or using the linear distance of the “nearest neighboring electrode” may result in inaccurate and/or distorted results.

As stated, it may be desirable to substitute the curved distance between electrodes for the linear distance in some example interpolation methods. Geodesic distances may be understood to be the shortest distance between two points in curved space. Therefore, calculating the geodesic distance between two electrodes may better approximate the distance between the two electrodes in curved space. An example method for calculating the geodesic distance may include creating a coarse triangular mesh between electrodes 24. The coarse triangular mesh may then be upsampled. The upsampled mesh may then be utilized to calculate the shortest distance between electrodes. Once the shortest distance between electrodes 24 has been calculated, the geodesic distance between electrodes 24 may be calculated. After generating the geodesic distances between electrodes 24, the geodesic distances may be substituted for the linear distance between electrodes 24.

While a triangular mesh may be useful, other geometries may be utilized. For example, the mesh may include other geometric shapes and/or configurations such as a polygon (e.g., having 4, 5, 6, 7, 8, 9, 10, or more sides), regular polygons, irregular polygons, etc.

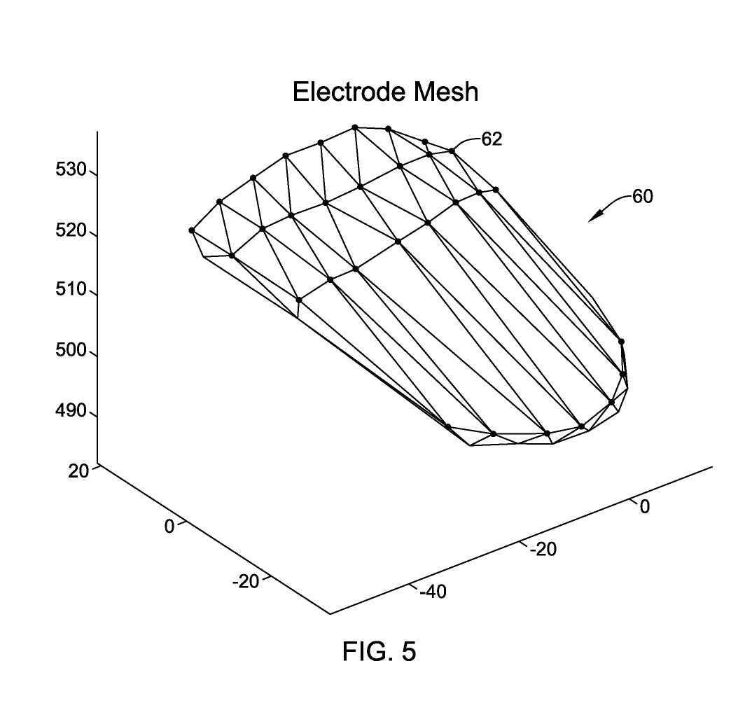

FIG. 5 illustrates a mesh 60 representing the three-dimensional arrangement of mapping electrodes 24 deployed in a non-uniform or non-spherical configuration. The mesh 60 may include interconnected nodes and/or vertices 62. Vertices 62 may be disposed at locations where mapping electrodes 24 are positioned. In at least some embodiments, the mesh 60 may take the form of a course triangular mesh. Creating a course triangular mesh may include approximating the geometry and/or the shape of a three-dimensional structure such as the three-dimensional arrangement of mapping electrodes 24. For example, a course triangular mesh may be designed to approximate the shape and physical relationships between electrodes 24 disposed on the basket structure 20 of a constellation catheter and/or similar sensing device deployed within a cardiac chamber of the heart. A triangular mesh may include a set of triangles that are drawn between the electrodes 24. Further, the three-dimensional configuration may include flat faces and straight edges and/or lines that connect electrodes 24 together by their common edges or corners. The corners of the triangular faces may be defined as vertices 62.

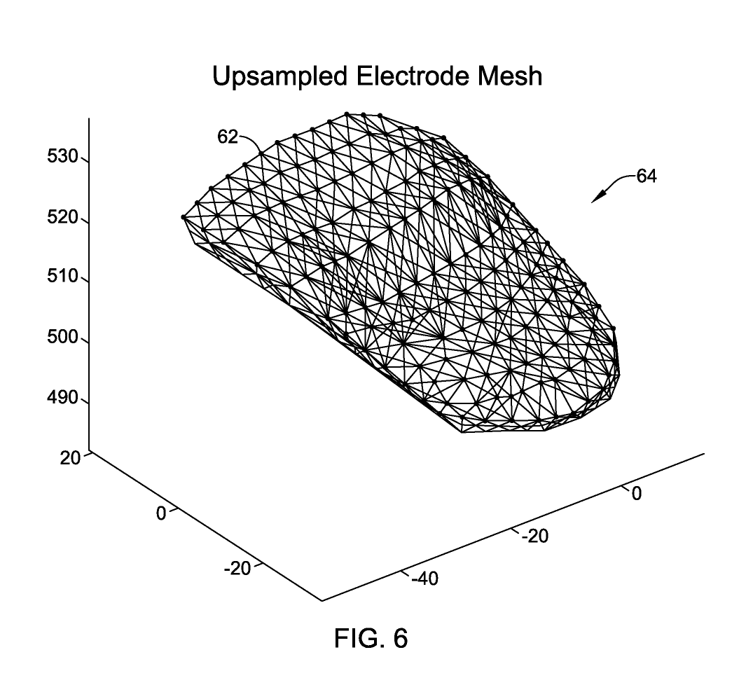

In at least some embodiments, it may desirable to further refine or “upsample” mesh 60. FIG. 6 illustrates a schematic upsampled mesh 64. The upsampled mesh 64 may include interconnected nodes and/or vertices 62. The upsampled mesh 64 may be generated from a course triangular mesh. Upsampling may include subdividing the triangles of the triangular mesh into additional triangles. The additional triangles may include flat faces and straight edges and/or straight lines connecting vertices 62 of the triangles.

The upsampled mesh 64 may be utilized to calculate the shortest distance between electrodes. For example, after the shortest distance between electrodes is calculated, the upsampled mesh 64 may be utilized to calculate the geodesic distances between electrodes. The geodesic distances may be substituted for the linear distance in an example interpolation method. For example, the geodesic distance between two electrodes may be substituted for the linear distance between the electrodes in RBF, Kriging or similar interpolation methods. Using geodesic distance estimations instead of linear distance approximations or assumptions may provide a more accurate estimate of the interpolated data points.

In at least some embodiments, one or more interpolation methods stated above may be incorporated, included, utilized, and/or integrated into processing system 32. Processing system 32 may be configured such that the interpolation method may be implemented to populate and/or fill in electrodes 24 having missing data on activation map 72. Further, processing system 32 may incorporate an “iterative” process to assess, populate and/or fill in electrodes 24 having missing data on activation map 72. The iterative process may cycle through determining an electrode 24 that has missing data, utilizing an interpolation method to estimate missing and/or inaccurate data and populating and/or filling in the missing data on the corresponding activation map 72. The processing system 32 may integrate and/or employ a feedback loop in the iterative process. For example, the processing system 32 may integrate and/or employ a feedback loop when interpolating, choosing, and/or assigning activation times and populating and/or filling in activation map 72. A feedback loop may be designed to permit an operator (e.g. physician, clinician) to select the number of iterations processing system 32 will implement to populate activation map 72. For example, a user (e.g. physician, clinician) may be able to input the number of iterations that processing system 32 will implement to populate activation map 72. It is further contemplated that processing system 32 may include a preset maximum number of iterations that it will implement when populating activation map 72.

The disclosed embodiments heretofore have focused on populating and/or estimating unknown and/or inaccurate data in an activation map. However, it is contemplated that the above methodologies may be utilized to estimate unknown and/or inaccurate data as it relates to any diagnostic display, data set, diagnostic visual representation, or the like. For example, the above methodologies may be utilized to estimate unknown and/or inaccurate data for a vector field map, isochronal map, or the like.

In at least some of the embodiments described above the disclosed methods assume analysis of sensed, collected, measured and transmitted electrical cellular data occurring during a single heartbeat and/or cardiac pulse. However, it is contemplated that any of the disclosed methods may be implemented across multiple beats or cardiac pacing time intervals. Further, data collected over multiple heartbeats may be analyzed using statistical methodologies and applied to the disclosed methods. For example, activation times may be collected over a series of heart beats and/or pulses. A statistical distribution of the collected activation times may be calculated, analyzed and incorporated into disclosed methods.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A medical device, comprising:
a catheter shaft with a plurality of electrodes coupled thereto;
a processor coupled to the catheter shaft, wherein the processor is capable of:
collecting a set of signals from the plurality of electrodes;
generating a data set from at least one of the set of signals, wherein the data set includes at least one known data point and one or more unknown data points;
determining a non-linear distance between the at least one known data point and the one or more unknown data points; and
assigning a value to at least one of the unknown data points.

2. The medical device of claim 1, wherein collecting the set of signals further includes sensing a change in electrical potential by any one of the plurality of electrodes.

3. The medical device of claim 2, further comprising identifying a threshold value corresponding to a minimum change in electrical potential by any one of the plurality of electrodes; and
wherein collecting the set of signals includes collecting only those signals that are above the threshold value.

4. The medical device of claim 1, wherein collecting the set of signals includes determining an activation time at one or more of the plurality of electrodes and wherein determining the activation time includes identifying a fiducial point corresponding to a change in electrical potential and determining a time latency between a reference point and the fiducial point.

5. The medical device of claim 1, wherein determining the non-linear distance between at least one known data point and one or more unknown data points includes creating a mesh of interconnected nodes between the known data points, the unknown data points or both the known and unknown data points.

6. The medical device of claim 5, wherein determining the non-linear distance between at least one known data point and one or more unknown data points includes upsampling the mesh of interconnected nodes.

7. The medical device of claim 1, wherein determining the non-linear distance between at least one known data point and one or more unknown data points includes determining a geodesic distance between the known data points, unknown data points or both the known and unknown data points.

8. The medical device of claim 1, wherein assigning a value to at least one of the unknown data points includes interpolating the at least one unknown data point, and wherein interpolating the at least one unknown data point includes utilizing the non-linear distance between the at least one known data point and the unknown data point.

9. The medical device of claim 8, wherein interpolating the at least one unknown data point includes a radial basis function interpolation.

10. The medical device of claim 9, wherein interpolating the at least one unknown data point further includes utilizing a geodesic distance in the radial basis function interpolation.

11. The medical device of claim 8, wherein utilizing the non-linear distance between the at least one known data point and the unknown data point further comprises using a geodesic distance between the at least one known data point and the unknown data point.

12. The medical device of claim 1, wherein assigning a value to at least one of the unknown data points includes assigning an activation time to at least one of the unknown data points.

13. The medical device of claim 12, wherein assigning an activation time to at least one of the unknown data points further comprises a radial basis function interpolation of activation times, wherein the radial basis function interpolation incorporates a geodesic distance between at least one known data point and one or more unknown data points.

14. The medical device of claim 1, further comprising generating a visual display of between at least one known data point, one or more unknown data points or both and wherein generating a visual representation includes creating an activation map, wherein the activation map further comprises a plurality of color indicators.

15. A medical device for mapping the electrical activity of the heart, comprising:
a catheter shaft coupled to a sensing element, wherein the sensing element includes a plurality of electrodes coupled thereto;
a processor coupled to the catheter shaft, wherein the processor is capable of:
collecting a set of signals from the plurality of electrodes;
generating a data set from at least one of the set of signals, wherein the data set includes at least one known data point and one or more unknown data points;
determining a non-linear distance between the at least one known data point and the one or more unknown data points;
interpolating the at least one unknown data point from the at least one known data point, wherein interpolating the at least one unknown data point includes utilizing the non-linear distance between the at least one known data point and the unknown data point; and
assigning a value to at least one of the unknown data points.

16. The medical device of claim 15, wherein determining the non-linear distance between at least one known data point and one or more unknown data points includes determining a geodesic distance between the known data points, unknown data points or both the known and unknown data points.

17. The medical device of claim 15, wherein interpolating the at least one unknown data point further includes utilizing a geodesic distance in a radial basis function interpolation.

18. The medical device of claim 15, wherein utilizing the non-linear distance between the at least one known data point and the unknown data point further comprises using a geodesic distance between the at least one known data point and the unknown data point.

19. The medical device of claim 15, wherein assigning a value to at least one of the unknown data points includes assigning an activation time to at least one of the unknown data points and wherein assigning an activation time to at least one of the unknown data points further comprises a radial basis function interpolation of activation times, wherein the radial basis function interpolation incorporates a geodesic distance between at least one known data point and one or more unknown data points.

20. A method of mapping the electrical activity of the heart, the method comprising:
advancing a catheter shaft with a plurality of electrodes coupled thereto into a chamber of the heart, wherein the catheter shaft is coupled to a processor, wherein the processor is capable of:
collecting a set of signals from the plurality of electrodes;
generating a data set from at least one of the set of signals, wherein the data set includes at least one known data point and one or more unknown data points;
determining a non-linear distance between the at least one known data point and the one or more unknown data points; and
assigning a value to at least one of the unknown data points.
